(19) United States
(12) Patent Application Publication
(10) Pub. No.: US 2026/0114780 A1
(43) Pub. Date: Apr. 30, 2026

(54) TISSUE STATE MEASURING APPARATUS

(71) Applicant: NATIONAL TSING HUA UNIVERSITY, Hsinchu (TW)

(72) Inventor: TING-WEI WANG, Hsinchu (TW)

(73) Assignee: NATIONAL TSING HUA UNIVERSITY, Hsinchu (TW)

(21) Appl. No.: 19/367,059

(22) Filed: Oct. 23, 2025

(30) Foreign Application Priority Data
Oct. 30, 2024 (TW) 113141708

(51) Int. Cl. A61B 5/277; A61B 5/279

(52) U.S. Cl. CPC A61B 5/277; A61B 5/279

(57) ABSTRACT

The tissue status measuring apparatus includes an electrode set and a control module coupled to the electrode set. The electrode set is configured to emit a first sensing electric field toward a dielectric area and to form a capacitive loop with the dielectric area. The control module includes a driving unit and a processing unit. The driving unit is configured to output a driving signal to the electrode set to output the first sensing electric field. The processing unit is configured to measure a loop capacitance value of the capacitive loop. In response to a tissue under test being located within the dielectric area, the loop capacitance value of the capacitive loop is a first measured capacitance value. In response to a difference between the first measured capacitance value and a baseline capacitance value being greater than an abnormality threshold, the processing unit determines that the tissue under test has an abnormal tissue location.

### TECHNICAL FIELD

The present invention relates to a tissue status measuring apparatus; in particular to, a non-contact tissue status measuring apparatus based on electric field measurement.

### BACKGROUND

By measuring tissue status (for example, hemorrhage, edema, or tissue-related status information) using medical instruments, the therapeutic needs of a subject are assessed. In existing medical systems, tissue status measurement is mostly performed using MRI or other large-scale medical equipment. Although large-scale medical equipment provides precise or accurate measurement results, such equipment does not offer convenience and immediacy. In addition, from a cost perspective, large-scale medical equipment cannot be widely deployed to the medical frontline. These limitations of large-scale medical equipment result in their inability to be effectively applied in point-of-care (POC) and other applications.

Accordingly, there is considerable room for advancement and improvement in tissue status measurement technologies for medical applications.

### SUMMARY

One objective of the present invention is to provide a tissue status measuring apparatus that enables portable and on-site medical applications.

Another objective of the present invention is to provide a tissue status measuring apparatus having a rapid and non-contact measuring mechanism, thereby achieving the purpose of point-of-care applications.

In an embodiment, the present invention provides a tissue status measuring apparatus. The tissue status measuring apparatus includes an electrode set and a control module coupled to the electrode set. The electrode set is configured to emit a first sensing electric field toward a dielectric area and to form a capacitive loop with the dielectric area. The control module includes a driving unit and a processing unit. The driving unit is configured to output a driving signal to the electrode set to output the first sensing electric field. The processing unit is configured to measure a loop capacitance value of the capacitive loop. In response to a tissue under test being located within the dielectric area, the loop capacitance value of the capacitive loop is a first measured capacitance value. In response to a difference between the first measured capacitance value and a baseline capacitance value being greater than an abnormality threshold, the processing unit determines that the tissue under test has an abnormal tissue location.

In an embodiment, in response to a reference tissue being located within the dielectric area, the loop capacitance value of the capacitive loop is a second measured capacitance value; and the processing unit obtains the baseline capacitance value according to the second measured capacitance value.

In an embodiment, the tissue under test is a brain measurement region; and in response to the first measured capacitance value being greater than the baseline capacitance value and the difference being greater than the abnormality threshold, the processing unit determines the abnormal tissue location as a hemorrhagic abnormality.

In an embodiment, the tissue under test is a brain measurement region; and in response to the first measured capacitance value being less than the baseline capacitance value and the difference being greater than the abnormality threshold, the processing unit determines the abnormal tissue location as an ischemic abnormality.

In an embodiment, the electrode set is further configured to emit a second sensing electric field toward the dielectric area; wherein the first sensing electric field has a first penetration depth, and the second sensing electric field has a second penetration depth.

In an embodiment, the electrode set includes a first electrode set configured to emit the first sensing electric field, and a second electrode set configured to emit the second sensing electric field.

In an embodiment, a spacing between a first electrode and a second electrode of the first electrode set is equal to a spacing between a third electrode and a fourth electrode of the second electrode set; and the length of the first electrode is different from that of the third electrode.

In an embodiment, the electrode set is an annular structure.

In an embodiment, the electrode set includes a first electrode and a second electrode, the first electrode and the second electrode forming one of: concentric circular type, spiral type, interdigital type, spiral interdigital type, or concentric interdigital type.

In an embodiment, the electrodes are disposed on a first surface of a first film, and a shielding film is disposed on a second surface of the first film.

In an embodiment, the driving signal has an output frequency; wherein an equivalent circuit of the electrode set has a reference inductance value; and the processing unit calculates the loop capacitance value according to a received frequency from the electrode set and the reference inductance value.

In an embodiment, the processing unit is further configured to generate a grayscale value and coordinate information according to the loop capacitance value; wherein, in response to a plurality of tissues under test being located within the dielectric area, the processing unit generates a plurality of measured grayscale values and a plurality of measured coordinate information; and the processing unit generates a tissue status map according to the plurality of measured grayscale values and the plurality of measured coordinate information.

In an embodiment, the processing unit calculates a baseline grayscale value of the tissue status map according to the baseline capacitance value.

In an embodiment, the processing unit further generates a plurality of tissue status maps corresponding to different depths, and generates a three-dimensional tissue status map according to the plurality of tissue status maps.

In an embodiment, the electrode set includes a plurality of electrodes, the plurality of electrodes being configured to form a plurality of capacitive loops with the dielectric area; and the processing unit is further configured to measure the loop capacitance value of each of the plurality of capacitive loops and generate corresponding grayscale values.

In an embodiment, the plurality of electrodes are integrated into a head-mounted structure.

By generating sensing electric fields through the electrode set, non-contact measurement is achieved and the sensing electric fields pass through the skin surface to measure deeper tissue status. The tissue status is estimated based on changes in capacitance values measured by the electrode set resulting from dielectric properties of different tissue states. Thus, a rapid and non-contact measuring mechanism is realized. Moreover, compared with large-scale medical equipment, the tissue status measuring apparatus, by adopting a configuration of the electrode set and the control module, reduces volume and manufacturing cost, thereby enabling portable and on-site medical applications

### BRIEF DESCRIPTION OF THE DRAWINGS

The accompanying drawings are presented to help describe various aspects of the present invention. In order to simplify the accompanying drawings and highlight the contents to be presented in the accompanying drawings, conventional structures or elements in the accompanying drawings may be drawn in a simple schematic way or may be omitted. For example, a number of elements may be singular or plural. These accompanying drawings are provided merely to illustrate these aspects and not to limit the invention.

FIGS. 1 to 2 are schematic diagrams of a tissue status measuring apparatus according to one embodiment of the present invention.

FIG. 3 is a schematic diagram illustrating the measurement of a loop capacitance value according to one embodiment of the present invention.

FIG. 4 is a schematic diagram illustrating a mutual-inductance structure forming a capacitive loop according to one embodiment of the present invention.

FIG. 5 is a schematic diagram illustrating a self-inductance structure forming a capacitive loop according to one embodiment of the present invention.

FIG. 6 is a schematic diagram of an electrode set outputting a plurality of sensing electric fields according to one embodiment of the present invention.

FIG. 7 is a schematic circuit diagram illustrating impedance parameter regulation of the electrode set according to one embodiment of the present invention.

FIG. 8 is a schematic diagram of an electrode set having a plurality of sub-electrode sets according to one embodiment of the present invention.

FIG. 9 is a schematic diagram of an electrode set outputting a plurality of sensing electric fields by adjusting spacings among the plurality of electrodes according to one embodiment of the present invention.

FIG. 10 is a schematic diagram of an electrode set outputting a plurality of sensing electric fields by adjusting layout patterns among the plurality of electrodes according to one embodiment of the present invention.

FIG. 11 is a schematic diagram of an electrode set having different electrode patterns according to one embodiment of the present invention.

FIG. 12 is a schematic diagram illustrating imaging according to measurement results in accordance with one embodiment of the present invention.

### DETAILED DESCRIPTION

Any reference to elements using terms such as “first” and “second” herein generally does not limit the number or order of these elements. Conversely, these names are used herein as a convenient way to distinguish two or more elements or element instances. Therefore, it should be understood that the terms “first” and “second” in the request item do not necessarily correspond to the same names in the written description. Furthermore, it should be understood that references to the first element and the second element do not indicate that only two elements are used or that the first element needs to precede the second element. Open terms such as “include”, “comprise”, “have”, “contain”, and the like as used herein mean including but not limited to.

The term “coupled” is used herein to refer to direct or indirect electrical coupling between two structures. For example, in an example of indirect electrical coupling, one structure may be coupled with another structure through a passive element such as a resistor, a capacitor, or an inductor.

In the present invention, the term such as “exemplary” or “for example” is used to represent “giving an example, instance, or description”. Any implementation or aspect described herein as “exemplary” or “for example” is not necessarily to be construed as preferred or advantageous over other aspects of the present invention. The terms “about” and “approximately” as used herein with respect to a specified value or characteristic are intended to represent within a value (for example, 10%) of the specified value or characteristic.

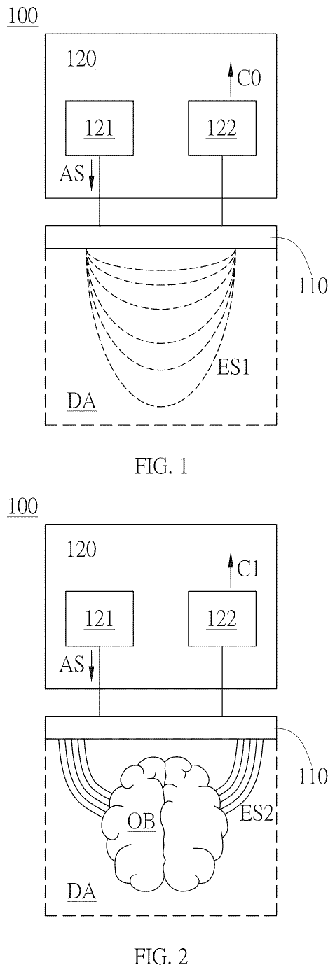

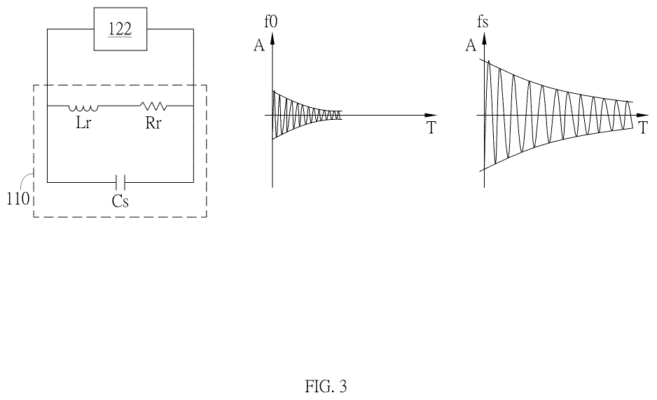

FIGS. 1 to 2 illustrate a tissue status measuring apparatus 100 according to one embodiment of the present invention. Referring to FIGS. 1 to 2, the tissue status measuring apparatus 100 includes an electrode set 110 and a control module 120 coupled to the electrode set 110. The electrode set 110 is configured to emit a first sensing electric field ES1 toward a dielectric area DA and to form a capacitive loop with the dielectric area DA. The control module 120 includes a driving unit 121 and a processing unit 122. The driving unit 121 is configured to output a driving signal AS to the electrode set 110 to output the first sensing electric field ES1. The processing unit 122 is configured to measure a loop capacitance value C0 of the capacitive loop. In response to a tissue under test OB being located within the dielectric area DA, the loop capacitance value of the capacitive loop is a first measured capacitance value C1. In response to a difference between the first measured capacitance value C1 and a baseline capacitance value being greater than an abnormality threshold (not shown in FIGS. 1 to 2), the processing unit 122 determines that the tissue under test OB has an abnormal tissue location.

In an embodiment, the processing unit 122 is, for example, constituted by a programmable processor such as a microprocessor, FPGA, ASIC, or SoC. In an embodiment, the processing unit 122 further includes modules such as analog-to-digital conversion, frequency measurement, or capacitance measurement components to perform control and measurement. In an embodiment, the control module 120 is a user device with programmable functionality, such as a computer, a smartphone, or a notebook computer, and the processing unit 122 is the processor and/or related peripheral components built inside the user device.

In an embodiment for measuring the loop capacitance value of the capacitive loop, the loop capacitance value or a variation of the loop capacitance value is obtained through changes in the frequency of the first sensing electric field ES1 in response to the tissue under test OB being located within the dielectric area DA. Specifically, referring to FIG. 3, the driving signal AS has an output frequency (f0). The equivalent circuit of the electrode set 110 includes a reference inductance value Lr and a reference resistance value Rr. When the tissue under test OB is located within the dielectric area DA, the loop capacitance value Cs of the capacitive loop will be changed, such that the resonant frequency (f1) of an equivalent RLC circuit formed by the loop capacitance value Cs, the reference inductance value Lr, and the reference resistance value Rr will also be changed. The processing unit 122 is configured to calculate the loop capacitance value Cs based on the frequency variation received from the electrode set 110 due to the change in the resonant frequency and the reference inductance value Lr according to the following formula:

$$fs = \frac{1}{2 π \sqrt{LrCs}}$$

However, the measurement of the loop capacitance value Cs disclosed by the present invention is not limited to this embodiment, and may also be performed by using electrical principles related to capacitance.

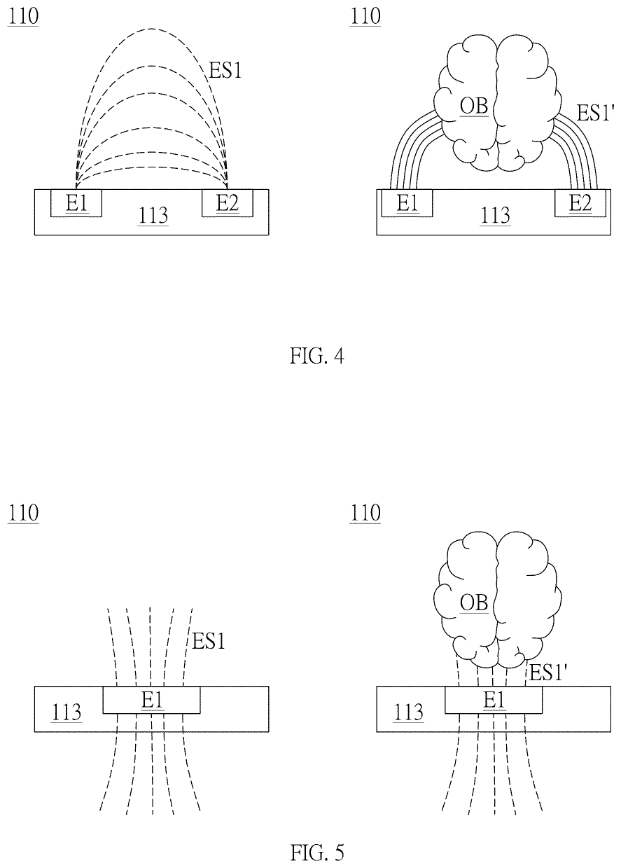

In an embodiment of the capacitive loop, the electrode set 110 and the dielectric area DA form the capacitive loop in a mutual-inductance structure. Specifically, referring to FIG. 4, the electrode set 110 includes a first electrode E1 and a second electrode E2 disposed on a substrate 113. The first sensing electric field ES1 is transmitted from one of the first electrode E1 and the second electrode E2 (for example, the first electrode E1), passes through the dielectric area DA, and is received by the other electrode (for example, the second electrode E2). Accordingly, the first electrode E1, the dielectric area DA, and the second electrode E2 form the capacitive loop. When the tissue under test OB is located within the dielectric area DA, the tissue under test OB will be polarized, thereby changing the first sensing electric field ES1′. The variation of the first sensing electric field ES1′ causes a change in the loop capacitance value Cs of the capacitive loop. Furthermore, the dielectric constant of the tissue under test OB also causes a change in the loop capacitance value Cs. At this time, the measured loop capacitance value Cs is the first measured capacitance value C1. Therefore, the difference between the first measured capacitance value C1 and the abnormality threshold is inferred as the measurement result corresponding to the status of the tissue under test OB. The abnormality threshold is determined by obtaining dielectric constants of tissues under various conditions through anatomical studies or simulations, and by defining the abnormality threshold according to the difference in dielectric constants between normal tissue and abnormal tissue.

In another specific embodiment of the capacitive loop, the electrode set 110 and the dielectric area DA form the capacitive loop in a self-inductance structure. Specifically, referring to FIG. 5, the electrode set 110 includes a first electrode E1 disposed on a substrate 113. The first electrode E1 emits the first sensing electric field ES1 and forms a capacitive loop with an object (for example, the tissue under test OB) within the first sensing electric field ES1. Similar to the mutual-inductance structure, when the tissue under test OB is located within the first sensing electric field ES1 and forms the capacitive loop with the first electrode E1, the dielectric constant of the tissue under test OB causes different responses in the loop capacitance value Cs of the capacitive loop. In the embodiment, the measured loop capacitance value Cs is the first measured capacitance value C1. Therefore, the first measured capacitance value C1 is inferred as the measurement result corresponding to the status of the tissue under test OB.

In an embodiment in which the tissue status measuring apparatus 100 infers the status of the tissue under test OB based on the first measured capacitance value C1, the tissue status measuring apparatus 100 may be used to measure an abnormal location and/or depth of hemorrhage or ischemia in the brain. Specifically, stroke is an acute cerebrovascular disease, and the timeliness of medical intervention has a significant correlation with the degree of recovery and extent of damage in a patient. In general, brain stroke is classified into ischemic stroke and hemorrhagic stroke. Ischemic stroke and hemorrhagic stroke respectively require different treatment methods to achieve better therapeutic effects, and the determination of treatment methods and the application within the golden time window will greatly affect treatment outcomes and recovery potential. Therefore, by outputting the first sensing electric field ES1 toward the brain of the subject through the tissue status measuring apparatus 100 and measuring the first measured capacitance value C1, the stroke location and/or depth can be quickly estimated, the type of stroke can be identified, and the most appropriate treatment can be administered to achieve the best therapeutic effect.

In this embodiment, according to current research in the field, the dielectric constant (εₙ) of normal brain tissue is approximately (43.22), the dielectric constant (εᵢ) of brain tissue affected by ischemic stroke is approximately (30), and the dielectric constant (εₕ) of brain tissue affected by hemorrhagic stroke is approximately (62). It should be noted that the above dielectric constant values are provided merely for illustrating the implementation of this embodiment, and the measurement of the present invention is not limited to these example values. The abnormality threshold may be selected based on the difference between the dielectric constant (εₙ) of normal brain tissue and the dielectric constant (εₕ) of hemorrhagic stroke brain tissue or the dielectric constant (εᵢ) of ischemic stroke brain tissue. By utilizing differences in dielectric constants of tissues in different conditions that lead to variations in capacitance values of the capacitive loop, and by setting an abnormality threshold to reduce the scope of misjudgment, the stroke location and/or depth can be rapidly estimated, and the type of stroke can be inferred so that the most appropriate treatment can be administered to achieve the best therapeutic effect.

However, the present invention is not limited to measuring the brain, nor is it limited to stroke type detection. In other words, the present invention effectively applies to the determination of tissue conditions based on differences in dielectric constants under various states. In one application embodiment, the present invention may be applied to hemorrhagic conditions that cannot be directly observed (for example, internal bleeding). More specifically, when a blood vessel surrounding a tissue ruptures but the blood does not flow out to the body surface and thus cannot be directly observed, measurement can still be performed using the tissue status measuring apparatus 100. The tissue surrounding the hemorrhage also exhibits a difference in dielectric constant compared with normal tissue. By means of the tissue status measuring apparatus 100, it is possible to quickly confirm whether a tissue is suffering from problems such as a hemorrhagic condition. In another application embodiment, the present invention is also applied to conditions in which differences in dielectric constants exist between the tissue under test and normal tissue due to gaseous conditions (for example, flatulence), solid conditions (for example, foreign bodies), or liquid conditions (for example, edema or hemorrhage).

Compared with large-scale medical equipment, the tissue status measuring apparatus 100 of the present invention adopts the configuration of the electrode set 110 and the control module 120. Both the electrode set 110 and the control module 120 are miniaturized and manufactured at reduced cost through optimized fabrication processes such as integrated circuit manufacturing, printed circuit board manufacturing, and molding. Therefore, the tissue status measuring apparatus 100 of the present invention achieves portability and on-site medical applications. For example, the tissue status measuring apparatus 100 may be equipped in frontline medical scenarios (e.g., ambulances or emergency response teams) for emergency situations. By using the tissue status measuring apparatus 100, rapid, non-contact, or non-invasive tissue status measurement is provided to patients, enabling quick interpretation of tissue conditions and the provision of the most appropriate treatment under current conditions. In this way, patients will be provided suitable treatment within the golden time of therapy, thereby improving treatment effectiveness and postoperative recovery.

In an embodiment, the tissue status measuring apparatus 100 may establish a baseline according to a reference tissue, thereby reducing measurement error or interference. Specifically, the tissue status measuring apparatus 100 may respond to a reference tissue (for example, normal brain tissue) located within the dielectric area DA. At this time, the loop capacitance value of the capacitive loop formed by the electrode set 110 and the reference tissue is a second measured capacitance value. The processing unit 122 obtains a baseline capacitance value according to the second measured capacitance value. For example, the baseline capacitance value may be equal to the second measured capacitance value or may be a value obtained through post-processing such as averaging or filtering of the second measured capacitance value. By establishing the baseline capacitance value, a capacitance value corresponding to the reference or normal tissue can be obtained. When abnormal tissue (for example, tissue affected by stroke) is located within the dielectric area DA, the first measured capacitance value C1 will differ from the baseline capacitance value. The state of the abnormal tissue can thus be inferred based on the difference between the first measured capacitance value C1 and the baseline capacitance value.

In the embodiment of establishing a baseline, the difference between the first measured capacitance value C1 and the baseline capacitance value may be used to infer the state of abnormal tissue, for example, to distinguish hemorrhagic abnormalities or ischemic abnormalities. For instance, in response to the first measured capacitance value C1 being greater than the baseline capacitance value and the difference being greater than the abnormality threshold, the processing unit 122 determines that the abnormal tissue location is a hemorrhagic abnormality. On the other hand, in response to the first measured capacitance value C1 being less than the baseline capacitance value and the difference being greater than the abnormality threshold, the processing unit 122 determines that the abnormal tissue location is an ischemic abnormality.

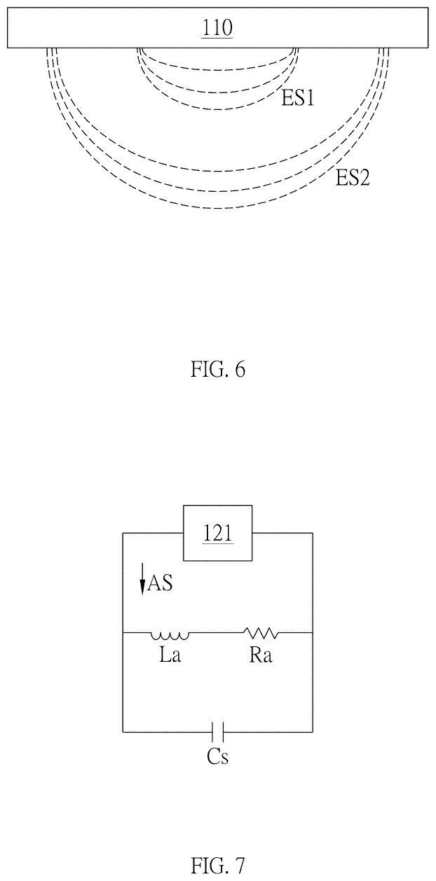

In an embodiment, the tissue status measuring apparatus 100 may perform measurements at different depths, thereby obtaining the depth of a tissue in an abnormal state. Specifically, referring to FIG. 6, the electrode set 110 is further configured to emit a first sensing electric field ES1 and a second sensing electric field ES2 (i.e., a plurality of sensing electric fields) toward the dielectric area DA. The first sensing electric field ES1 has a first penetration depth, and the second sensing electric field ES2 has a second penetration depth. For example, by adjusting the frequency or field pattern of the first sensing electric field ES1 and the second sensing electric field ES2, the first sensing electric field ES1 and the second sensing electric field ES2 may have different frequencies to achieve different penetration depths. Through the use of a plurality of sensing electric fields with different penetration depths, the tissue status measuring apparatus 100 may perform measurements at different depths, thereby obtaining the depth or longitudinal range of tissue in an abnormal state.

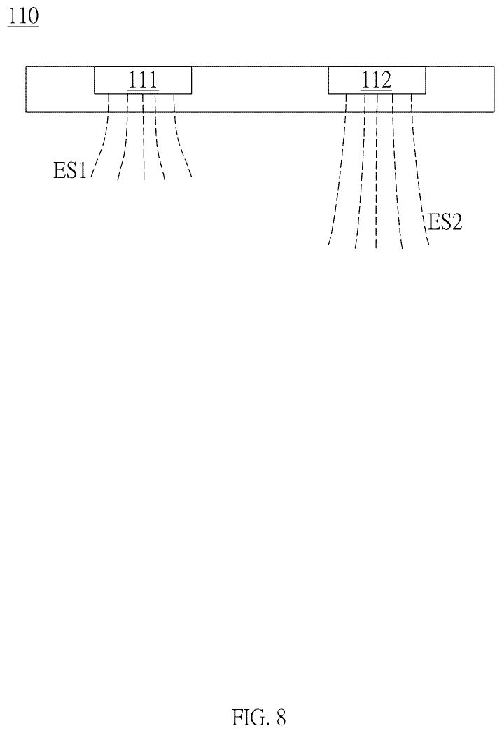

In an embodiment of adjusting the frequency of the sensing electric fields, regulation may be performed through the electrode set 110. Referring to FIG. 7, the electrode set 110 may be coupled to tunable passive components (for example, an adjustable resistor Ra or an adjustable inductor La). Such tunable passive components may, for example, change the impedance of the electrode set 110 by means of switching. When the driving unit 121 provides the driving signal AS to the electrode set 110, the electrode set 110 has different resonant frequencies due to different impedance values, thereby outputting the first sensing electric field ES1 and the second sensing electric field ES2 at different frequencies. On the other hand, the electrode set 110 may output the first sensing electric field ES1 and the second sensing electric field ES2 at different frequencies through multiple sub-electrode sets. Referring to FIG. 8, the electrode set 110 includes a first electrode set 111 configured to emit the first sensing electric field ES1, and a second electrode set 112 configured to emit the second sensing electric field ES2. The first electrode set 111 and the second electrode set 112 are configured to have different impedance parameters (i.e., different RLC equivalent circuits). When the driving unit 121 provides the driving signal AS to the first electrode set 111 and the second electrode set 112, the first electrode set 111 and the second electrode set 112 will have different frequency responses due to the different impedance parameters, thereby outputting the first sensing electric field ES1 and the second sensing electric field ES2 at different frequencies.

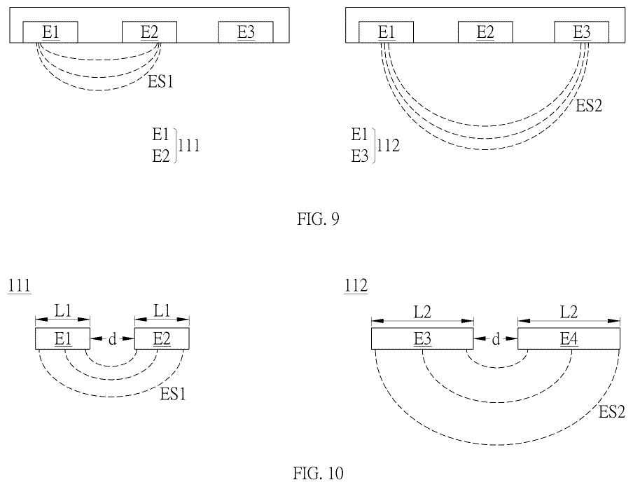

In an embodiment of adjusting the field patterns of the sensing electric fields, the first electrode set 111 and the second electrode set 112 may be configured to have different field patterns by adjusting respective electrode patterns or spacings. For example, referring to FIG. 9, the electrode set 110 may include multiple electrodes (for example, E1-E3), and the distances between the electrodes may be adjusted, for example, by switching, to output different sensing electric fields. For instance, electrodes E1 and E2 may be classified as the first electrode set 111, and electrodes E1 and E3 may be classified as the second electrode set 112; however, the invention is not limited thereto. As an example of adjusting electrode patterns, the spacing d between the first electrode E1 and the second electrode E2 of the first electrode set 111 may be equal to the spacing d between the third electrode and the fourth electrode of the second electrode set 112. Furthermore, the length L1 of the first electrode E1 may be different from the length L2 of the third electrode. By adjusting the electrode patterns (for example, cross-sectional area) or spacings, the electrode set 110 generates different sensing electric fields corresponding to different depths. It should be noted that FIG. 8 is provided only to illustrate that the field patterns of the electrode set 110 may be adjusted through electrode patterns or spacings, and is not intended to limit the means for adjusting the field patterns of the sensing electric fields of the present invention.

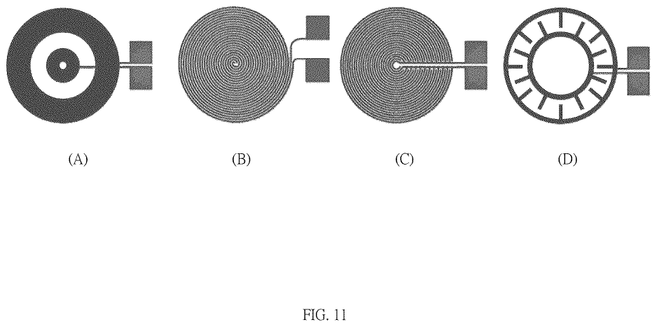

In an embodiment of the electrode set 110, the first electrode E1 and the second electrode E2 of the first electrode set 111 may be configured in various electrode patterns. For example, referring to FIG. 11, the first electrode E1 and the second electrode E2 of the electrode set 110 may be configured as annular electrodes. The first electrode E1 and the second electrode E2 may form concentric circular type (A), spiral interdigital type (B), interdigital type (C), or concentric interdigital type (D), but are not limited thereto. By adjusting the width, cross-sectional area, and/or spacing between the first electrode E1 and the second electrode E2, the first sensing electric field ES1 may be shaped into different field patterns. Different field patterns may be used to adjust the penetration depth of the first sensing electric field ES1 or the sensitivity of the electrode set 110.

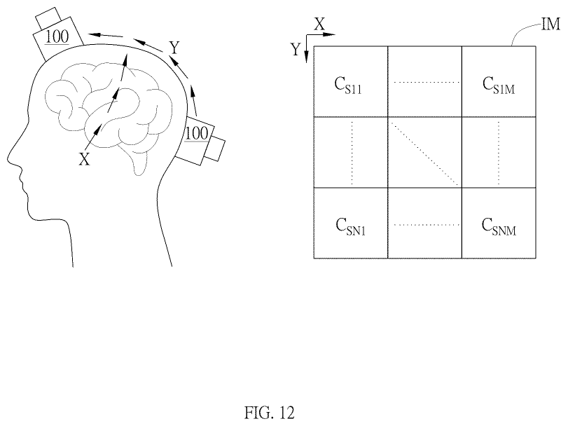

In an embodiment, the tissue status measuring apparatus 100 may perform imaging based on the measurement results. Referring to FIG. 12, the processing unit 122 is further configured to generate grayscale values and coordinate information according to the loop capacitance values. In response to the tissue under test OB being located within the dielectric area DA, the processing unit 122 generates a plurality of measured grayscale values and a plurality of measured coordinate information. The processing unit 122 generates a tissue status map IM according to the plurality of measured grayscale values and the plurality of measured coordinate information. Specifically, the grayscale values may be converted from changes in the loop capacitance value or the measured loop capacitance values Cs₁₁-Cs_{NM} (for example, into grayscale values ranging from 0 to 255). The processing unit 122 may further calculate a baseline grayscale value of the tissue status map according to the baseline capacitance value as described in the foregoing embodiments. On the other hand, the coordinate information (1,1)-(N,M) may be generated by planar scanning (for example, scanning directions X and Y shown in FIG. 12) or by means such as an electrode array to provide multiple coordinate information corresponding to the measurement results. Specifically, in an embodiment using an electrode array, the electrode set 110 may include multiple electrodes arranged to extend along direction X and direction Y and configured to form multiple capacitive loops with the dielectric area DA. The processing unit 122 is further configured to measure the loop capacitance value of each of the multiple capacitive loops and generate corresponding grayscale values.

In the embodiment of using multiple electrodes, the multiple electrodes may further be integrated into a wearable structure. For example, the multiple electrodes may be integrated into a head-mounted structure. By wearing the head-mounted structure 200, the multiple electrodes may correspond to all target measurement positions and each have respective coordinate information. The processing unit 122 may integrate and image based on the measurement results of the multiple electrodes. The wearable structure simplifies steps such as scanning, making the coordinate information less affected by scanning methods. The wearable structure also effectively integrates the multiple electrodes to avoid inaccurate imaging results caused by differences in electrode placement.

In an embodiment of imaging, the tissue status measuring apparatus may generate multiple tissue status maps corresponding to different depths through measurements at different depths, and generate a three-dimensional tissue status map according to the multiple tissue status maps. Measurement at different depths may be based on different frequencies or field patterns as proposed in the foregoing embodiments, or may alternatively be achieved through three-dimensional scanning methods. Thus, a three-dimensional tissue status map is generated. With such imaging information, an operator can more easily to determine the range, depth, or location of abnormal tissue, and improves diagnostic accuracy through cross-referencing with normal tissue.

The aforementioned description of the present invention is provided to enable a person of ordinary skill in the art to make or implement the present invention. Various modifications to the present invention will be apparent to a person skilled in the art, and the general principles defined herein is applied to other variations without departing from the spirit or scope of the present invention. Therefore, the present invention is not intended to be limited to the examples described herein, but is to be in accord with the widest scope consistent with the principles and novel features of the invention herein.

What is claimed is:

1. An apparatus for measuring tissue status, comprising:
an electrode set configured to emit a first sensing electric field toward a dielectric area and to form a capacitive loop with the dielectric area; and
a control module coupled to the electrode set, including:
a driving unit configured to output a driving signal to the electrode set to output the first sensing electric field; and
a processing unit configured to measure a loop capacitance value of the capacitive loop;
wherein, in response to a tissue under test being located within the dielectric area, the loop capacitance value of the capacitive loop is a first measured capacitance value; and
wherein, in response to a difference between the first measured capacitance value and a baseline capacitance value being greater than an abnormality threshold, the processing unit determines that the tissue under test has an abnormal tissue location.

2. The apparatus for measuring tissue status of claim 1, wherein in response to a reference tissue being located within the dielectric area, the loop capacitance value of the capacitive loop is a second measured capacitance value; and wherein the processing unit obtains the baseline capacitance value according to the second measured capacitance value.

3. The apparatus for measuring tissue status of claim 1, wherein the tissue under test is a brain measurement region; and wherein in response to the first measured capacitance value being greater than the baseline capacitance value and the difference being greater than the abnormality threshold, the processing unit determines the abnormal tissue location as a hemorrhagic abnormality.

4. The apparatus for measuring tissue status of claim 1, wherein the tissue under test is a brain measurement region; and wherein in response to the first measured capacitance value being less than the baseline capacitance value and the difference being greater than the abnormality threshold, the processing unit determines the abnormal tissue location as an ischemic abnormality.

5. The apparatus for measuring tissue status of claim 1, wherein the electrode set is further configured to emit a second sensing electric field toward the dielectric area; and wherein the first sensing electric field has a first penetration depth, and the second sensing electric field has a second penetration depth.

6. The apparatus for measuring tissue status of claim 5, wherein the electrode set comprises a first electrode set configured to emit the first sensing electric field, and a second electrode set configured to emit the second sensing electric field.

7. The apparatus for measuring tissue status of claim 6, wherein a spacing between a first electrode and a second electrode of the first electrode set is equal to a spacing between a third electrode and a fourth electrode of the second electrode set; and wherein a length of the first electrode is different from a length of the third electrode.

8. The apparatus for measuring tissue status of claim 1, wherein the electrode set is an annular structure.

9. The apparatus for measuring tissue status of claim 8, wherein the electrode set comprises a first electrode and a second electrode, the first electrode and the second electrode forming one of: concentric circular type, spiral type, interdigital type, spiral interdigital type, or concentric interdigital type.

10. The apparatus for measuring tissue status of claim 1, wherein the driving signal has an output frequency; wherein the equivalent circuit of the electrode set has a reference inductance value; and wherein the processing unit calculates the loop capacitance value according to a received frequency from the electrode set and the reference inductance value.

11. The apparatus for measuring tissue status of claim 1, wherein the processing unit is further configured to generate a grayscale value and coordinate information according to the loop capacitance value; wherein, in response to a plurality of tissues under test being located within the dielectric area, the processing unit generates a plurality of measured grayscale values and a plurality of measured coordinate information; and wherein the processing unit generates a tissue status map according to the plurality of measured grayscale values and the plurality of measured coordinate information.

12. The apparatus for measuring tissue status of claim 11, wherein the processing unit calculates a baseline grayscale value of the tissue status map according to the baseline capacitance value.

13. The apparatus for measuring tissue status of claim 11, wherein the processing unit further generates a plurality of tissue status maps corresponding to different depths, and generates a three-dimensional tissue status map according to the plurality of tissue status maps.

14. The apparatus for measuring tissue status of claim 11, wherein the electrode set comprises:
a plurality of electrodes configured to form a plurality of capacitive loops with the dielectric area; and
wherein the processing unit is further configured to measure the loop capacitance value of each of the plurality of capacitive loops and generate corresponding grayscale values.

15. The apparatus for measuring tissue status of claim 14, wherein the plurality of electrodes are integrated into a head-mounted structure.
